(19) United States
(12) Patent Application Publication
(10) Pub. No.: US 2008/0177204 A1
(43) Pub. Date: Jul. 24, 2008

(54) Apparatus and method for detection of cervical dilation during labor

(76) Inventor: Bernard Greenberg, Alpharetta, GA (US)

Correspondence Address: CARLTON FIELDS, PA, 1201 WEST PEACHTREE STREET, 3000 ONE ATLANTIC CENTER, ATLANTA, GA, 30309, US

(21) Appl. No.: 11/657,321

(22) Filed: Jan. 24, 2007

(51) Int. Cl. A61B 5/107

(52) U.S. Cl. 600/588

(57) ABSTRACT

An instrument for measuring a dilatation of cervix uteri during the first stage of labor. The instrument has two elongated legs pivotally connected to each other. Each leg has a proximal end and a distal end; the top end has an attaching device that can be attached to an inner surface of the cervix. The lower end of one leg is secured to a scale and the lower end of the other leg can move along this scale as the cervix dilates.

### FIELD OF THE INVENTION

The present invention relates to medical devices and more particularly, to an instrument and corresponding method for measuring dilatation of cervix uteri in childbirth.

### BACKGROUND OF THE INVENTION

During childbirth, it is customary in the medical management of labor to measure the dilatation of cervix uteri by inserting two fingers into the birth canal to bring them into contact with the edge of the orifice of the dilated cervix uteri and to evaluate the degree of dilatation manually. Such measurements are often repeated up to 10 times during the labor. Each vaginal intrusion by the obstetrician's gloved hand causes discomfort to the patient and increases the chance of introduction of bacteria that can cause infection in both the mother and newborn.

One of the major risks during childbirth is the introduction of bacteria into the birth canal and uterus by these repetitive exams of cervix uteri. This risk is particularly accentuated if the membranes have ruptured. If the bacteria are introduced during childbirth, it may cause sepsis in the newborn baby or post-partum pelvic infection in the mother. Both sepsis and pelvic infection are major causes of mother and newborn morbidity and mortality. Repeated insertion of the fingers or devices increase the risk of introducing harmful bacteria.

Several devices have been made to improve the dilatation measuring process. These devices merely attempt to improve the accuracy of measuring cervical dilation but do not reduce discomfort and risks of infection from repetitive exams. Most devices are placed on a physician or nurse's hand and fitted to their fingers. When the physician inserts his fingers into the birth canal, he extends his fingers, which are fitted with a measuring device, to the sides of the cervix and reads the measurement. The measurement device may improve the accuracy of diagnosis. However, these types of devices, since they still rely on repetitive manual examinations of the mother's cervix, do not reduce the discomfort for the mother or reduce the risk for the fetus.

Therefore, it is desirable to have a medical device for measuring cervical dilation during childbirth while minimizing the possibility of infection from repeated insertion of items into the birth canal and reducing the discomfort of the mother from the repeated insertions into her vaginal area, and to such a device that the present invention is primarily directed.

### SUMMARY OF THE INVENTION

Briefly described, a medical device is disclosed in the present application. The medical device has two extended arms, with one end of each arm attach to the inside region of the cervix and other end of the arms providing a dilation indication about the aperture of the cervix. In one embodiment, there is provided an apparatus for measuring the cervical dilation during the labor. The apparatus includes a first leg and a second leg pivotally secured together at an intermediate point to allow relative pivoting movement, and each leg has a first end and a second end. The apparatus can also include a first attaching device secured to the first end of the first leg and a second attaching device secured to the first end of the second leg. After the first attaching device and the second attaching device are attached to opposite lips of the cervix in the early phase of labor, continued dilation of the cervix causes the second end of the second leg to move away form the first leg, thus providing an indication of dilation of the human cervix. Additionally, a visual indicator may be attached to the second end of each leg, thus providing a visual indication of the dilation of the cervix. Alternatively, a scale arm may be connected to the second end of the first leg and the dilation of the cervix then causes the second end of the second leg to move away from the first leg and over the scale arm.

In another embodiment, there is provided a method for measuring dilation of a cervix during the initial stage of labor using a measuring device with two pivotally connected legs. The method includes attaching the first leg of the measuring device to an inside lip of the cervix, attaching the second leg of the measuring device to an opposition position on the inside lip of the cervix, reading a distance between the first leg and the second leg, and obtaining a measurement of dilation based on the distance. Alternatively, if a scale arm is attached to the first leg and the second leg is capable of moving over the scale arm, reading of a distance separating the first leg and the second leg provides the measurement of dilation.

Since the device accurately measures cervical dilation as labor progresses, no additional manual digital exams are needed. This eliminates the discomfort laboring women typically experience, and greatly reduces the chance of maternal or newborn infection

Additional features and advantages of the invention will be readily apparent from the following detailed description and appended claims when taken in conjunction with the accompanying drawings.

### BRIEF DESCRIPTION OF THE DRAWING

FIG. 1 is an anatomical view of the pelvic region during the process of labor showing use of one embodiment of a measuring device in accord with the present invention.

FIG. 2 depicts a measuring device according to one embodiment of the invention.

FIG. 3 depicts a measuring device according to an alternative embodiment of the invention.

FIG. 4 illustrates an exemplary process for using the measuring device.

FIG. 5 illustrates an alternative embodiment of a measuring device.

### DETAIL DESCRIPTION OF THE INVENTION

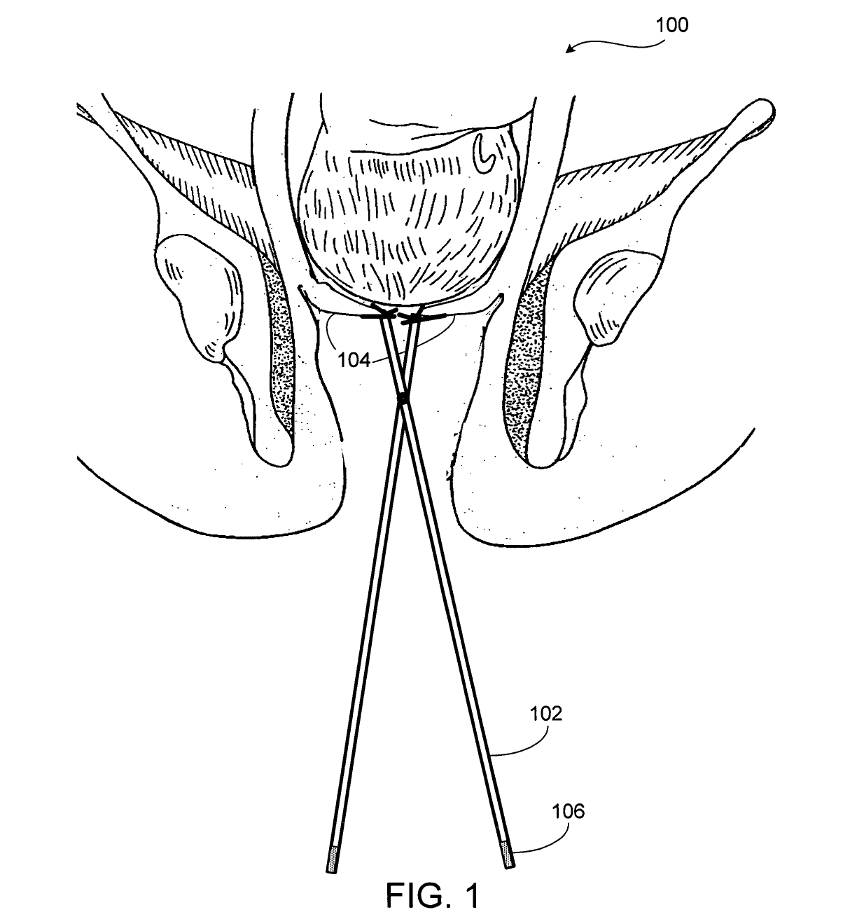

The measuring device according to one embodiment of the invention enables monitoring of dilation of the cervical region during the initial stage of labor and reduces the risk of bacterial infection. FIG. 1 is an illustration 100 of the pelvic region during the process of labor. There is shown a measuring device 102 with two elongated legs, inserted and attached to the inner region of cervix 104. The end of each elongated leg, which is outside of a woman's body, is preferably equipped with a clearly visible visual indicator 106. The visual indicator 106 is preferably made from a light reflective material. The distance between the two visual indicators provides a visual indication of the dilation of the cervix. The two elongated legs have a length preferably between 6 to 8 inches and are capable of performing scissors-like movement. The attachment is such that a portion of the measuring device is exposed outside of the vaginal area and visible to physician or nurse. The measuring device 102 has a size that is small enough that does not impede a mother's movement, i.e., the mother may be able to walk with the measuring device attached while waiting for the cervical region to dilate. On the other hand, the measuring device should be large enough that it enables a nurse to read the measurement that reflects the dilation of the birth canal. The measuring device 102 is preferably made from metal, plastic, card stock material, or some other light and bio-friendly material. The measuring device 102 preferably is packed individually and disposable after a single use, minimizing the possibility for infection.

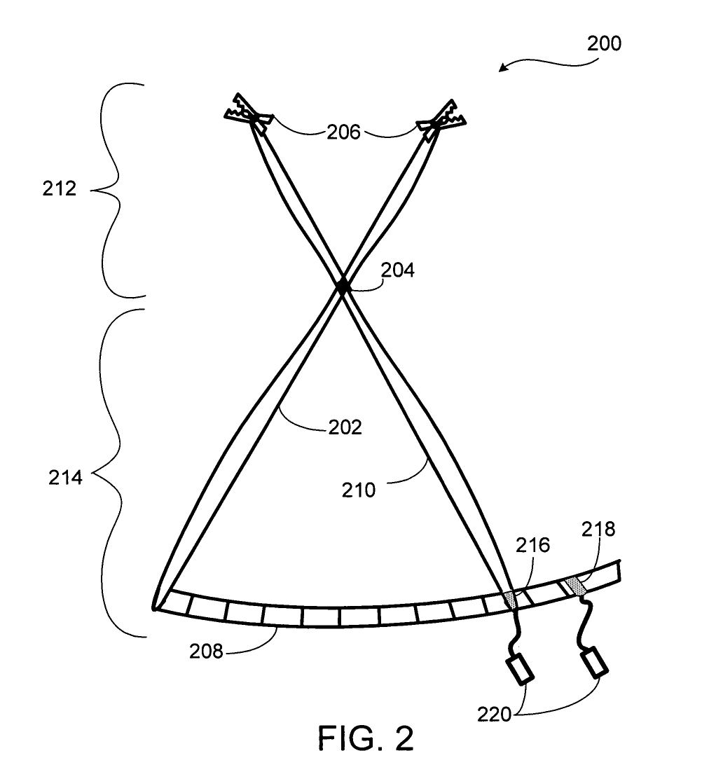

FIG. 2 illustrates a measuring device 200 according to one embodiment of the invention. The measuring device 200 has two elongated legs 202, 210 connected to each other at an intermediate point through a pivot or a screw 204 such that relative movement between two elongated legs is possible. Each elongated leg has a top (proximal) end and a lower (distal) end and the top end is attached to an attaching device 206 that can be a clip, an adhesive tape, a clamp, a helical attaching pin, a socket with a receiving cavity for receiving opposite lips or rims of the cervix, or any attaching means that enable attachment of two elongated legs of the measuring device to the cervix. In this embodiment, the lower end of one elongated leg 202 is attached to a measuring scale arm 208 that is marked with graduation marks. The measuring scale arm 208 may have an arcuate shape and the lower end of another elongated leg 210 can move freely, concentrically along the measuring scale arm 208. Once the top end of each elongated leg is attached to the opposite lips of the cervix, the position where the elongated leg 210 rests indicates the approximate distance between the top ends of two elongated legs. Two elongated legs 202, 210 are attached through the pivot 204, which divides each elongated leg into two portions, upper portion 212 and lower portion 214. The lower portion 214 may be longer or shorter than the upper portion 212; preferably, the lower portion 214 is longer than the upper portion 212. However, mere separation of the second ends of the first and second legs can be sufficient to provide a visual indication of the state of dilation.

Optionally, a piece of electrical contact 216 can be placed at the lower portion 214 of the leg 210 that moves over the measuring scale arm 208, a slidable piece of electrical contact 218 may be placed anywhere along the measuring scale arm 208. Each of these electrical contacts may be a metallic contact and connected to an electrical connector 220, which may be attached to a monitoring device (not shown). If the monitoring device is used, when the dilatation of the cervix reaches a predefined dimension, causing the two metallic contacts to contact each other, the monitoring device can cause an alarm to sound or a notification to occur, thus alerting the nursing staff.

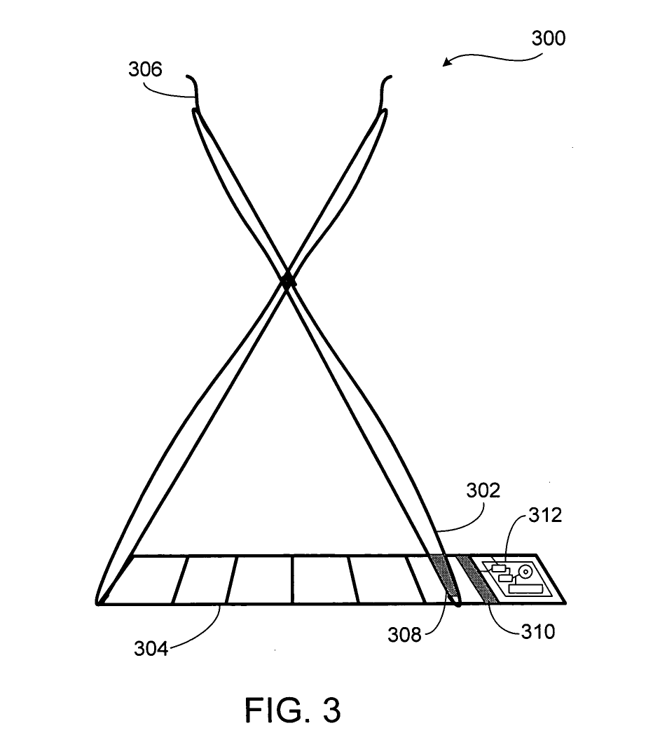

FIG. 3 illustrates an alternative embodiment 300 of the measuring device 302. The measuring device 302 is attached to a measuring scale arm 304 that is straight. The graduation marks are placed on the measuring scale arm 304 proportionally to the distance between the top ends of two elongated legs. The measuring device 302 is also equipped with adhesive tapes 306 that are capable of attaching to the cervix. Optionally, the measuring scale arm 304 may be constructed with an embedded electronic monitoring device 312 that includes a contact detection circuit, an audio device, and a built-in battery. The embedded electronic monitoring device 312 will sound when the measuring device 302 causes two metallic (electrical) contacts 308, 310 to close the contact detection circuit.

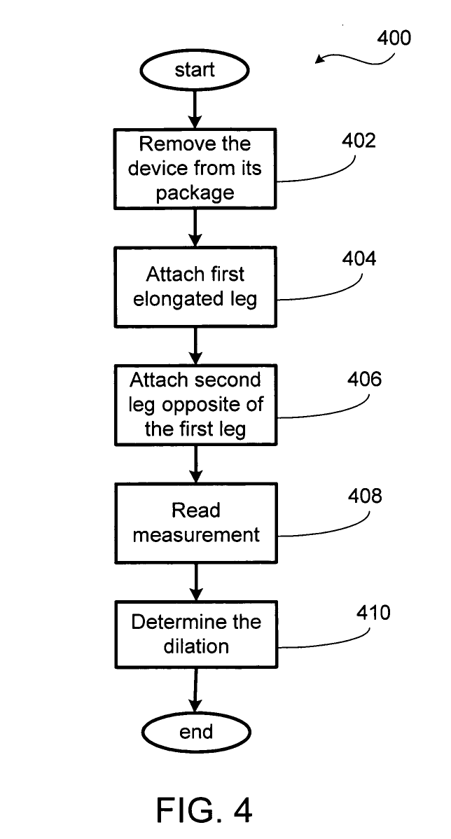

FIG. 4 illustrates a method of measuring dilation of the cervical region in accordance with one embodiment of the present invention with the use of the above-described instrument in the following manner. After an expecting mother arrives at a hospital in preparation for delivering a baby, at the first stage of labor, a physician or a nurse removes a measuring device from its package, step 402, and attaches two elongated legs of the measuring device to the opposite sides of the cervix, steps 404, 406, preferably at 3 and 9 o'clock positions. The device may be applied to the cervix either before or after rupture of the membranes. After the measuring device is securely attached to the cervix, the expecting mother may still be able to move around, if she chooses. Periodically, the physician or nurse will read the measuring scale arm, step 408, and determine the dilation of the cervix, step 410. During further dilatation of cervix uteri, the top ends of two elongated legs move apart from one another and the lower ends of the elongated legs also move apart from one another. The distance between two top ends can be easily determined by reading the marks that separate two lower ends. Once the device is in place, no additional manual digital exams are needed. Thus the method described above reduces discomfort to the expecting mother caused by the traditional repeated digital insertions. The use of a disposable and sterile measuring device with just one vaginal exam further reduces the possibility of introduction of bacteria or other contaminants during the labor when compared to the current method of repetitive vaginal exams. The measuring device may be removed when the cervix is fully dilated or when the expecting mother starts to push.

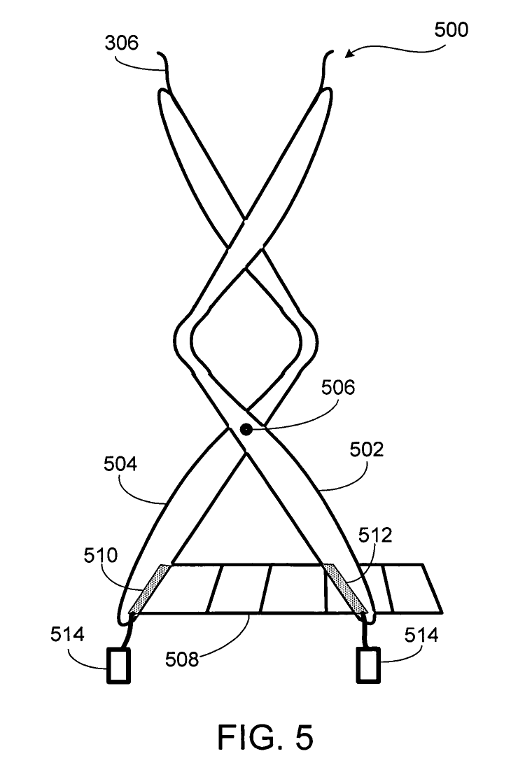

FIG. 5 depicts a measuring device 500 according to an alternative embodiment. The measuring device 500 includes two elongated legs 502, 504 connected through a pivot point 506. Each elongated leg is generally V-shaped. The lower end of one elongated leg 504 is secured to a scale arm 508 while the lower end of other elongated leg 502 can move freely along the scale arm 508. Because two uniquely shaped elongated legs 502, 504 are secured at the pivot point 506, when the top ends of these legs are attached to opposite sides in the cervix, the lower ends are separated from each other. As the cervix dilates, the lower ends actually approaches to each other. The opposing surfaces of two elongated legs are preferably flat, such that even when the top ends move away from each other beyond the normal, expected distance, the lower end of the elongated leg 502 may move “off-scale” and not hinder the dilation of the cervix.

Optionally, each elongated leg 502, 504 may include an electric contact, 510, 512, which maybe connected with an external connector 514 for connecting with a monitor (not shown). The measuring device 500 may be connected to the monitor through the external connectors 514. As the cervix dilates, one elongated leg 502 moves toward other elongated leg 504, and when the two electric contacts 510, 512 touch each other, the monitor may sound an alarm or otherwise notify the nursing staff.

It will be understood that each of the elements described above may also find a useful application in other types of construction. The invention is not intended to be limited to the details shown, since various modifications may be made without departing from the spirit of the present invention. Without further analysis, the foregoing will so fully reveal the gist of the present invention that others can, by applying current knowledge, readily adapt it for various applications without omitting features that, from the standpoint of prior art, fairly constitute essential characteristics of the generic or specific aspects of this invention.

What is claimed is:

1. An apparatus for measuring the cervical dilation during the first stage of labor, comprising:
a first and a second legs pivotally secured together by a pivot at an intermediate point to allow relative pivoting movement, each leg having a first end and a second end;
a first attaching device secured to the first end of the first leg; and
a second attaching device secured to the first end of the second leg,
wherein, after the first attaching device and the second attaching device being attached to opposite lips of a cervix, dilation of the cervix causing the second end of the second leg to move away from the second end of the first leg, thus providing a visual indication of dilation of the cervix.

2. The apparatus of claim 1, wherein the first and second legs being made from a plastic material.

3. The apparatus of claim 1, wherein the first and second attaching devices being made from a plastic material.

4. The apparatus of claim 1, wherein the first and second attaching devices being made from a metallic material.

5. The apparatus of claim 1, wherein the first and second legs being made from a card stock material.

6. The apparatus of claim 1, wherein the first and second attaching devices include adhesive.

7. The apparatus of claim 1, wherein the first and second attaching devices being clips.

8. The apparatus of claim 1, wherein the scale arm further being marked with graduation marks.

9. The apparatus of claim 1, wherein the scale arm further having an arcuate form and being concentric in relation to the pivot.

10. The apparatus of claim 1, wherein the dilation of the cervix causing the second end of the second leg to move closer to the second end of the first leg.

11. The apparatus of claim 1, further comprising a scale arm connected to the second end of the first leg.

12. The apparatus of claim 1, further comprising two visual indicators, one visual indicator being attached to the second end of each leg.

13. The apparatus of claim 1, wherein each leg further comprising a metallic contact at the second end.

14. The apparatus of claim 13, wherein each metallic contact further being connected to a connector for connecting to a monitor.

15. The apparatus of claim 13, further comprising a monitor device in communication with the metallic contacts on the second end of each leg.

16. A method for measuring dilation of a cervix during the initial stage of labor using a measuring device with pivotally a first leg and a second leg, comprising the steps of:
attaching the first leg of the measuring device to an inside lip of the cervix;
attaching the second leg of the measuring device to an opposition position on the inside lip of the cervix;
reading a distance between the first leg and the second leg; and
obtaining a measurement of dilation based on the distance read.

17. The method of claim 16, wherein the measuring device further includes a scale arm attached to the first leg and the second leg being capable of moving in a path over the scale arm, further comprising the steps of:
reading a position of the second leg over the scale arm; and
obtaining a measurement of dilation based on the position read.

18. The method of claim 16, further comprising the step of generating a notification when a distance between the first leg and the second leg reaches a predefined value.

19. An apparatus for measuring the cervical dilation during the first stage of labor, comprising:
a first and a second elongated means pivotally secured together by a pivot at an intermediate point to allow relative pivoting movement, each elongate means having a first end and a second end;
a first attaching means secured to the first end of the first elongate means; and
a second attaching device secured to the first end of the second elongated means,
wherein, after the first attaching means and the second attaching means being attached to opposite lips of a cervix, dilation of the cervix causing the second end of the second elongated means to move away from the second end of the first elongated means, thus providing an indication of dilation of the cervix.

20. The apparatus of claim 19, further comprising a measuring means connected to the second end of the first elongate means.
